(19) United States
(12) Patent Application Publication
(10) Pub. No.: US 2024/0350251 A1
(43) Pub. Date: Oct. 24, 2024

(54) STENT-GRAFT DEVICE WITH CONFORMING CONNECTOR AND METHODS OF USING THE SAME

(71) Applicant: Major Medical Devices, LLC, New York, NY (US)

(72) Inventor: Andrew Samuel KERR, New York, NY (US)

(21) Appl. No.: 18/684,377

(22) PCT Filed: Aug. 15, 2022
(86) PCT No.: PCT/US2022/040328

§ 371 Date: Feb. 16, 2024

Related U.S. Application Data
(60) Provisional application No. 63/233,380, filed on Aug. 16, 2021.
(60) Provisional application No. 63/290,800, filed on Dec. 17, 2021.

(51) Int. Cl. A61F 2/07; A61F 2/966

(52) U.S. Cl. CPC A61F 2/07; A61F 2/966; A61F 2220/0075

(57) ABSTRACT

Stent-graft devices, systems and methods having a flexible connection between a stent and a graft. The stent can have stent apices, and at least one of the stent apices forms a receptacle. A connecting ring is mounted to the graft and has connecting ring protrusions adapted to be received respectively by the receptacles. The connecting ring protrusions can be flexibly retained respectively by the receptacles so that the connecting ring can move with respect to the stent.

All references cited herein, including but not limited to patents and patent applications, are incorporated by reference in their entirety.

### BACKGROUND

An aneurysm is an abnormal enlargement or bulge in a blood vessel. Aortic aneurysms can cause embolization into branch vessels, aortic thrombosis, and aortic rupture. Damaged blood vessels can be treated or repaired by surgery or by endovascular graft placement.

The aorta is the largest artery in the body. More particularly, the aorta originates from the left ventricle of the heart, extends into the abdomen and bifurcates into the two iliac arteries. Arterial aneurysms (AA) can occur in every part of the aorta and its branches. Sabiston Textbook of Townsend, et al., Surgery: The Biological Basis of Modern Surgical Practice 20th Edition, pp. 1722-1753 (2017).

AAs typically are repaired with open surgery or endovascular aneurysm repair (EVAR). EVAR is touted as a minimally invasive procedure that utilizes stent-graft devices to repair an AA. A stent-graft device is a combination device that includes a stent portion and a graft portion connected to each other such that they can be deployed together to repair damage to a blood vessel.

A stent is typically an expandable metal lattice device inserted into a blood vessel and expanded to open a constricted, damaged or occluded blood vessel. In addition to opening the blood vessel, a stent can provide a rigid structural support to prevent the blood vessel from re-closing. Stents often are used together with balloon angioplasty.

A prosthetic graft is a medical device that can be used to replace or repair a diseased blood vessel. The graft can be made of a synthetic material (e.g., ePTFE, polyester) that can be expanded to approximate the diameter of the blood vessel in need of repair. The graft material provides a blood-tight seal such that the graft can support normal blood flow without leakage.

The graft of a stent-graft device can provide a substantially blood-tight seal, and the stent provides the support structure to prevent the stent-graft device from dislodgment under the pressure of normal blood flow. The combination of the stent with the prosthetic graft can be used to hydraulically isolate an aneurysm when positioned across the neck of the aneurysm. However, it is important for the stent-graft device to be deployed in the blood vessel in a manner that maintains a blood-tight seal and avoids the development of endoleaks. The target anatomy can present a challenge for delivering a stent-graft device because the angle or curvature of the blood vessel can vary between the location where the stent is deployed and the location where the graft is deployed.

An endovascular aneurysm repair (EVAR) procedure for infrarenal aortic aneurysm typically requires one puncture or incision in each of the femoral arteries followed by maneuvering the constrained stent-graft device in an upward, cephalad direction (femoral approach) into position in the infrarenal aorta over a guidewire constrained in an introducer sheath. A portion of the stent-graft device is deployed in the infrarenal aorta. Next, the stent-graft device is positioned and deployed in one iliac artery. Another stent-graft device is then positioned and deployed in the other iliac artery second via the puncture or incision in the other femoral artery. Such procedures rely on inserting and deploying stent-graft devices from below the diaphragm of a subject or patient.

As noted above, the standard EVAR procedure requires bilateral femoral punctures (i.e., one puncture or incision in each femoral artery) for arterial access, in addition to making fine positioning movements for the stent-graft device from a femoral approach orientation (from below the diaphragm). While superior to open surgery, EVAR, as described above, requires patient recovery from the bilateral femoral punctures used for access. Patients often remain at bedrest for several days, require pain medication, and thereby incur additional costs (e.g., hospital stay, medication, loss of time at work).

A need exists for improved stent-graft systems and methods that can be deployed in blood vessels in a manner that conforms to the configuration of the blood vessel in a manner that reduces or eliminates endoleaks, as compared to currently available stent-graft devices.

### SUMMARY

Stent-graft systems and methods described herein provide and utilize a stent-graft device having a flexible connection between the stent and graft. The stent-graft device can comprise a stent having plural stent apices, and one or more of the stent apices may comprise a receptacle. The stent-graft device also can comprise a connecting ring having connecting ring apices corresponding respectively to the stent apices. In some instances, the ring apices comprise protrusions adapted to be received respectively by the receptacles of the stent. In this aspect, the protrusions of the ring can be retained flexibly by a corresponding one of the receptacles, and the connecting ring can move with respect to the stent. In other instances, the stent apices can have the protrusions and the ring apices can have the receptacles. This alternate embodiment still permits flexible retention of the protrusions in the receptacles so that the connecting ring can move with respect to the stent.

Some aspects of the stent-graft systems described herein provide low profile stent-graft devices configured to be introduced/inserted in a blood vessel located above the diaphragm of the patient and deployed in a “top down” approach. Exemplary systems and methods for adjusting the placement of stent-graft devices in a target blood vessel (e.g., infrarenal aorta, juxtarenal aorta, pararenal aorta, thoracic aorta, or suprarenal aorta) after an initial deployment are also provided. Further aspects describe a centering device for use in centering the deployed location of stent-graft devices in a target blood vessel.

One aspect described herein is directed to a stent-graft system having a first stent, and a main graft body wherein the stent-graft system can be configured to be inserted through a single arterial puncture or incision in an insertion site blood vessel located above a diaphragm of a patient.

Further aspects provide a stent-graft system (e.g., an endograft deployment system) for deploying an endograft in a target blood vessel of a patient or subject. In some instances of the stent-graft system, the endograft can be deployed from below or above the diaphragm of a patient or subject, namely in a cephalad direction or in a caudad direction. The endograft deployment system can have an outer tube comprising a central inner member comprising an endograft, a carrier tube comprising a tether wire and/or a pusher catheter with a hub through which tubes, wires, tethers and the like can pass in a known manner. The tether wire can have a caudad end and a more cephalad portion (i.e., end closest to the head or top of the body). The endograft deployment system can have a top stent surrounding the central inner member, wherein the top stent comprises a plurality of hooks having a plurality of receptacles and a plurality of sutures.

Aspects described herein also provide a stent-graft system for repair of an aneurysm in a target blood vessel. This aspect of the disclosed stent graft system comprises a main graft body and a sealing stent at least partially disposed in the main graft body. This stent-graft system can be configured to be inserted through a single arterial puncture or incision in an insertion site blood vessel located above a diaphragm of a patient. Thus, the main-graft body can be configured to be inserted through a single arterial puncture or incision in an insertion blood vessel located above a diaphragm of a patient.

Further aspects described herein provide methods and devices for positioning a main graft body of a stent-graft system in a target blood vessel (e.g., infrarenal aorta, juxtarenal aorta, pararenal aorta, thoracic aorta, or suprarenal aorta) of a subject by advancing a main graft body delivery system to a target location in the target blood vessel. The main graft body delivery system can include the main body graft and a centering device. The main graft body can be positioned in the target blood vessel in a first position at the target location and it can be determined if the first position of the main graft body is centered in the target blood vessel at the target location. The main graft body can be repositioned in the target blood vessel if it is determined that the first position is not sufficiently centered. This aspect also can enable inserting the main graft body through a single arterial puncture or incision in an insertion blood vessel located above a diaphragm of a patient.

Aspects described herein also provide a stent-graft system comprising a top stent having positioning receptacles and a main graft body. The top stent and the main graft body may be in a substantially end-to-end configuration. The top stent and the main graft body can be disposed around an inner member. The stent-graft system may include a snare tube comprising a snare loop. A first end of the snare loop can be disposed in the snare tube. A second end of the snare loop can be disposed from the snare tube, through the positioning receptacles, around the inner member, and into the snare tube. The snare tube can be parallel to the inner member and insertion/removal of the inner member and the snare tube can be controlled from an end of the above-described outer tube or hub that remains external of the patient. The stent-graft system can be configured to be inserted through a single arterial puncture or incision in an insertion site blood vessel located above a diaphragm of the patient.

### BRIEF DESCRIPTION OF DRAWINGS

FIG. 1A is a schematic illustration of an exemplary stent-graft device having a ball and socket connection between the stent and connecting ring, and FIG. 1B is an enlarged depiction of one embodiment of the ball and socket joint;

FIG. 2A is a schematic illustration of an exemplary stent-graft device having a ball and socket connection between the stent and connecting ring with an optional bar to permit greater flexibility between the stent and connecting ring and FIG. 2B is an enlarged illustration of the ball and socket joint;

FIG. 3A is a schematic illustration of an alternative configuration of a stent-graft device having a single or double ball and socket with bars on each side, and FIG. 3B is an enlarged illustration of the ball and socket connection and the bars;

FIG. 4A is a schematic illustration of an exemplary stent-graft device with a ball and socket connection joint between the stent and the connecting ring with the device being deployed in the center of the lumen of a blood vessel, as shown more clearly in FIG. 4B;

FIG. 5 is a schematic illustration of an exemplary top view of a connecting ring and exemplary connectors disposed around the connecting ring; and

FIG. 6 is a schematic illustration of an exemplary cross section of a connector having a ball connected to a bar disposed in a socket.

FIG. 7 is a schematic illustration of an exemplary stent-graft device with three ball and socket connection joints arranged in series between an apex of the stent and a corresponding apex the connecting ring.

FIG. 8 is a schematic illustration of the exemplary stent-graft device of FIG. 7 and showing the stent-graft device deployed in a blood vessel and adapting to an angular alignment at the place of deployment.

FIG. 9 is a schematic illustration of the exemplary stent-graft device that uses struts to connect the stent apices to the ring apices.

FIG. 10 is a schematic illustration of the stent-graft device with a pusher catheter for positioning the stent-graft device in the blood vessel.

### DETAILED DESCRIPTION

Stent-graft systems and methods are provided herein to improve the introduction, positioning, and deployment of stent-graft devices for repair of aneurysms with reduced risk of rupture. It is understood that stent-graft devices in accordance with aspects described herein can be used to repair aneurysms in any suitable blood vessel.

A typical stent-graft device provides a connection between the stent and graft portions of the stent-graft device. For example, a connecting ring can be disposed in one end portion of the graft. The connecting ring can have connecting locations disposed around the connecting ring and the connecting locations can be engaged with mating connecting locations on an end of the stent. The connection between the stent and the graft typically is designed to form a blood-tight seal with the wall of the blood vessel to avoid the development of endoleaks following deployment of the stent-graft device. The known configurations for achieving a blood-tight seal with the wall of the blood vessel also result in the connection between the stent and the graft being relatively inflexible with very little bend.

If such a stent-graft device is deployed in a portion of a blood vessel with a variable target anatomy (e.g., one portion having suprarenal angle relative to another portion), the relatively inflexible stent-graft device will not be able to conform to the blood vessel in a manner that forms a blood-tight seal. Such a configuration leaves a patient exposed to increased risk of developing an endoleak.

To address this problem, aspects described herein provide one or more flexible joints between the connecting ring and the stent. For example, the flexible joint can comprise one or more gimbals or balls deployed on one of the ring and the stent and a corresponding socket or receptacle on the other of the ring and the stent. It is understood that the gimbal or ball portion of the connector can have any suitable geometry selected to be received by a corresponding receptacle on the other portion. The gimbal or ball portion can be deployed on the stent and the corresponding receptacle can be deployed on the connecting ring or vice versa. The connecting ring can be disposed, for example, at one axial end of the graft. As described herein, such a configuration permits the stent (or graft) portion to be deployed in a portion of the blood vessel having a different angle (e.g., suprarenal angle) with respect to the graft (or stent) while still forming and maintaining a blood-tight seal with the blood vessel wall because the connection between the two portions of the stent-graft device can rotate and adapt to the target anatomy of the blood vessel.

Thus, the graft can, for example, be deployed in an orientation parallel to the infrarenal aorta. Without the flexible, conforming connection, a high suprarenal angle between the stent and graft would force the top of the graft away from one wall of the aorta and increase the risk of forming a type 1 endoleak. The flexible connection (e.g., a gimbal/ball and socket) would permit the graft to conform uniformly to the aorta wall.

In one aspect, a first stent-graft device having a flexible connection between a stent and a graft is provided. The stent-graft device can comprise a stent having plural stent apices, wherein one or more of the stent apices comprises plural receptacles. The stent-graft device also can comprise a connecting ring associated with the graft and having connecting ring protrusions corresponding to the stent apices. In some instances, the connecting ring apices have protrusions adapted to be received respectively by the receptacles. In this aspect, the connecting ring protrusions can be retained flexibly by the receptacle and the connecting ring can move with respect to the stent.

In some aspects of the first stent-graft device, one or more tethers can be used in place of the connecting ring to connect the stent to the graft. For example, the tethers can be attached flexibly to the graft wall. Alternatively, the tethers can be used in addition to the connecting ring to provide further connection to the graft.

In some aspects of the first stent-graft device, plural graft fixation tethers are provided. A first end of each of the graft fixation tethers in this embodiment is fixed to one or more locations on the connecting ring, and a second end of each of the graft fixation tethers in this embodiment is fixed to the graft (e.g., graft wall). The graft fixation tethers can, for example, distribute axial forces placed on the graft.

In some aspects of the first stent-graft device, the graft fixation tethers can extend from the connecting ring to one or more stents (e.g., z stent) associated with or embedded in the graft material. In this aspect, the graft fixation tethers can, for example, distribute axial forces to the one or more stents associated with or embedded in the graft material.

The term “fixed,” as used herein, refers to a connection between the graft fixation tether and the connecting ring and/or the graft fixation tether and the graft. The connection can be any suitable type of connection (e.g., flexible, rigid) for a given application.

The term “tether” refers to loop(s) or similar structures that can be used, for example, to attach the stent or a portion of the stent to the graft or a connecting ring associated with (e.g., embedded in or attached to) the graft. A tether can be made from any suitable suture material (e.g., ePTFE) or thin metallic wire and can be rigid or flexible. A tether can also be embedded in, for example, a graft wall.

The term “stent” refers to a mesh or lattice tube structure made of, for example, super-elastic nitinol thin wire or laser cut from tubing (or any other suitable material, for example, titanium, or chromium cobalt alloy), that can be inserted into a blood vessel in a constrained state and deployed in an unconstrained state. In some aspects, an end of the stent comprises a plurality of apices comprising receptacles for receiving a plurality of protrusions (e.g., gimbal/ball or similar) disposed at corresponding positions on a connecting ring.

In some instances, the connecting ring comprises plural ring apices adapted to receive the respective receptacles. The connecting ring can be disposed in the main graft body. The connectors and receptacles can be made integral with stent (e.g., spot welded into position).

The term “connecting ring” refers to a structure (e.g., ring) adapted to connect to the stent and to be disposed in graft. The connecting ring can also be referred to as an annular ring. The connecting ring connects the stent to the graft and provides for primary sealing. In some instances, the stent and graft are in a substantially end-to-end configuration. In some aspects, the connecting ring has barbs or hooks to achieve the connection between the connecting ring and the graft.

In some aspects, the blood vessel or target blood vessel is selected from the group consisting of an infrarenal aorta, a juxtarenal aorta, a pararenal aorta, a thoracic aorta, or a suprarenal aorta.

It is to be understood that any suitable flexible protrusion-receptacle system can be employed to bear a majority of the axial loading forces between the stent and the connecting ring and to provide a connection that permits relatively free rotation of the connecting ring with respect to the stent.

The term “end-to-end configuration” refers to the connection of a component of the stent graft to another component at its margin (e.g., side by side, or end to end). An end-to-end configuration can avoid overlapping of components (i.e., coaxial) in the stent-graft introducer sheath. An overlapped or coaxial configuration of components requires a larger diameter introducer sheath, a bigger hole in the blood vessel, and potentially a bigger vessel for introducing the stent-graft. Exemplary configurations disclosed herein with an end-to-end configuration enable a smaller diameter stent-graft device to be used.

The term “end-to-end configuration” with respect to the stent-graft systems and devices described herein, can also refer to a circumstance where the wall thickness of the connecting ring disposed in the main graft body is less than the wall thickness at a connection between the first stent and the graft or, in some instances, the connecting ring when the plurality of receptacles receives the plurality of protrusions.

Aspects described herein provide a second stent-graft device having a flexible connection between a stent and a graft. The stent may have plural stent apices. One or more of the stent apices may comprise plural receptacles, such as 3-12 apices. A connecting ring of this aspect is associated with the graft. The connecting ring can have plural connecting ring protrusions corresponding to the stent apices. The connecting ring protrusions are adapted respectively to be received by the receptacles. The connecting protrusions can be retained flexibly by the receptacles. In some aspects, each of the connecting ring protrusions is adapted to bend substantially in one plane along its longitudinal axis.

The term “bend” can refer to movement of a connecting ring protrusion to move freely or to move in one direction and retract partially or fully back to an initial position due to, for example, flexibility and tensile strength of the connecting ring protrusion.

The term “substantially in one plane” refers to movement that is 50, 60, 70, 90, or 100% within a single plane (e.g., left to right, right to left, up and down, down and up, dorsal to ventral, ventral to dorsal, medial to lateral, or lateral to medial, etc.).

Of course, the stent-graft device disclosed herein can be used when there is no bend relative to the longitudinal axis or a very small bend relative to the longitudinal axis, such as in a range of 5-10°.

In some aspects of the second device, the one plane contains the longitudinal axis of the stent and/or the graft.

In further aspects of the second device, the connecting ring protrusions are constrained respectively in the receptacles such that the connecting ring protrusions are capable of moving substantially in one plane with respect to the stent and the graft.

The term “constrained,” as used herein refers to limiting the movement of the connecting ring protrusions such that the movement is in one plane with respect to the stent and the graft.

In one aspect of the second stent-graft device, the one plane extends from medial to lateral, lateral to medial, or lateral to lateral with respect to the stent and the graft. For example, the one plane can be from one side of the device to another side of the device. In another aspect, the plane is not from dorsal to ventral or ventral to dorsal.

In some instances, the connecting ring protrusions are constrained by channels dispose respectively in the receptacles. For example, a channel can be formed to constrain movement from a medial to lateral and lateral to medial plane within a connecting ring receptacle such that the connecting ring protrusion can only bend or move substantially within the channel (e.g., the plane defined by the channel).

In a further aspect of the second stent-graft device, the connecting ring is adapted to apply radial force to a blood vessel when the stent-graft device is deployed in the blood vessel. For example, radial force that is applied by the deployed stent with respect to the inner wall of the blood vessel can be transferred through a connecting ring protrusion (e.g., bar) to the connecting ring. The radial force applied by the connecting ring may help to position the stent-graft device in the blood vessel. The radial force applied by the connecting ring also may urge the graft radially outward against the blood vessel to help prevent endoleaks.

The connecting ring can be made of a metal, metal alloy, polymer, composite, or any other medically acceptable material having sufficient density or tensile strength to apply a radial force to the inner wall of the blood vessel.

In a further aspect of the second stent-graft device, the connecting ring protrusions further comprise bars. The term “bars” refers to a material (e.g., a metal, metal alloy, polymer, composite, or any other medically acceptable material) having a cross-section of any suitable shape (e.g., rectangle, square, rhombus, cylinder, triangle, symmetrical, and non-symmetrical). In addition, a “bar” can have varying density or tensile strength at its outer portions and middle portion.

In another aspect of the second stent-graft device, one or more of the bars is capable of transferring radial force from the stent to the connecting ring when the stent-graft device is deployed in a blood vessel. The connecting ring can optionally transfer radial force exerted by the connecting ring through the bar to the stent.

In a further aspect of the second stent-graft device, the bars comprise a material selected from the group consisting of a metal, metal alloy, polymer, composite, or any other medically acceptable material.

Aspects described herein provide a third stent-graft device having a flexible connection between a stent and a graft. The third stent-graft device can comprise a stent having plural stent apices and a connecting ring associated with the graft. The connecting ring can have bars corresponding respectively to the stent apices. The bars of some embodiments are connected flexibly to the stent apices. One or more of the bars may be adapted to move substantially in one plane with respect to the stent and the graft.

In one aspect of the third stent-graft device, the one plane extends from medial to lateral, lateral to medial, or lateral to lateral with respect to the stent and the graft.

One or more of the bars of the third stent-graft device may be constrained such that each bar is capable of moving substantially in one plane with respect to the stent and the graft.

One or more of the bars of the third stent-graft device may be constrained by one or more channels disposed between the stent and the connecting ring.

One or more of the bars of the third stent-graft device may be configured to transfer radial force from the stent to the connecting ring when the stent-graft device is deployed in a blood vessel.

The connecting ring of some embodiments of the third stent-graft device is adapted to apply radial force to a blood vessel when the stent-graft device is deployed in the blood vessel.

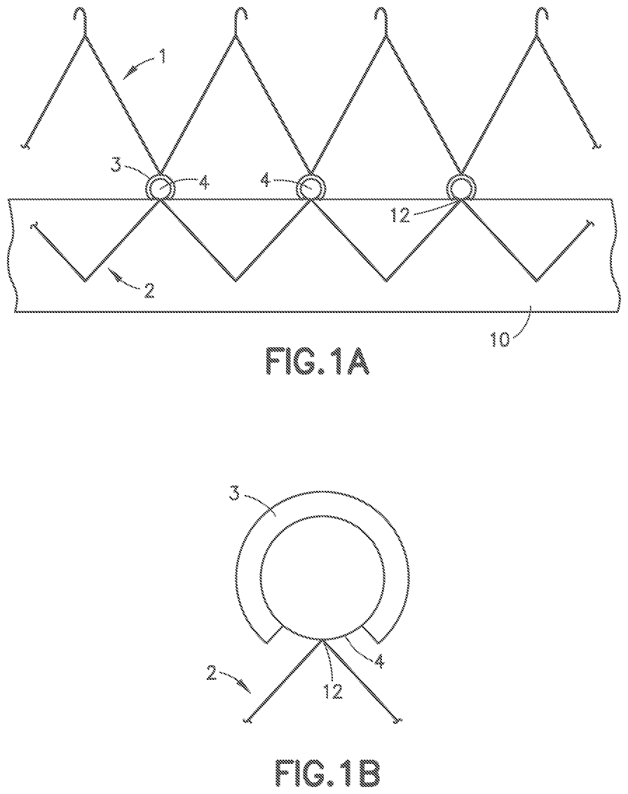

FIGS. 1A and 1B show an exemplary stent-graft device utilizing a ball and

socket connection between a stent 1 and a connecting ring 2. The connecting ring 2 of this embodiment is disposed at least partly in an axial end of a graft 10, and barbs or hooks 2b can pe provided on the connecting ring 2 for connecting the ring 2 to the wall of the aorta or other blood vessel. As shown in FIGS. 1A and 1B, a stent 1 is connected to connecting ring 2 using a socket 3 disposed on an apex 11 of the stent 1. A ball 4 is disposed on an apex 12 of the connecting ring 2 and is adapted to fit into the socket 3. The ball 4 is retained in the socket 3, but is free to rotate at least in certain directions in the socket 3. For example, the socket 3 may be configured to permit the ball 4 to rotate about an axis that extends parallel to the axis of the stent 1 and/or the axis of the connecting ring 2 or about an axis that is aligned angularly (e.g. perpendicular) to the axis of the stent 1 and/or the axis of the connecting ring 2. It is understood that the socket 3 could be attached to the connecting ring 2 and the ball 4 could be attached to stent 1. It is also understood that the ball 4 can have any suitable geometry that is adapted to be retained in the socket 3. In other words, the ball 4 is not required to be a sphere but could be a cube or hook, for example. In FIG. 1A, the connection between the socket 3 and the ball 4 is at a position beyond the end of the graft 10. In other embodiments, the connection between the socket 3 and the ball 4 is at a position at least partly within the graft 10

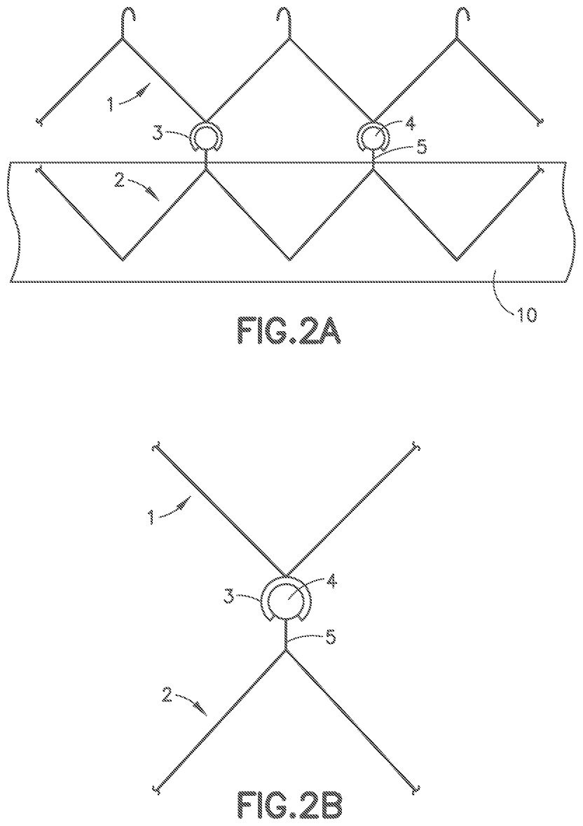

FIGS. 2A and 2B illustrate an alternative aspect a stent-graft device that further comprises a bar 5 disposed between ball 4 and the connecting ring 2. Increasing the distance between the connecting ring 2 and the stent 1 can provide even greater movement between stent 1 and connecting ring 2, if desired. Alternatively, the bar 5 can be disposed between ball 4 and the stent 1 with the socket 3 attached to connecting ring 2. In another aspect, a bar can be used to connect socket 3 to either the stent 1 or the connecting ring 2 and a bar 5 can be used to connect the ball 4 to either the stent 1 or the connecting ring 2.

In one aspect, the strength of the connection between the socket 3 and ball 4 is sufficient to prevent the connection ring 2 and the stent 1 from separating during or after deployment of the stent-graft device. In one aspect, the strength of the connection between the socket 3 and the ball 4 is at least 10, 20, 30, 40, 50, 60, 70, 80, 90, 100, 110, 120, 130, 140, 150, 160, 170, 180, 190, or 200 Newtons. In one aspect, the strength of the connection between the socket 3 and the ball 4 is at least 10 Newtons. In another aspect, the strength of the connection between the socket 3 and the ball 4 is between 100 and 200 Newtons

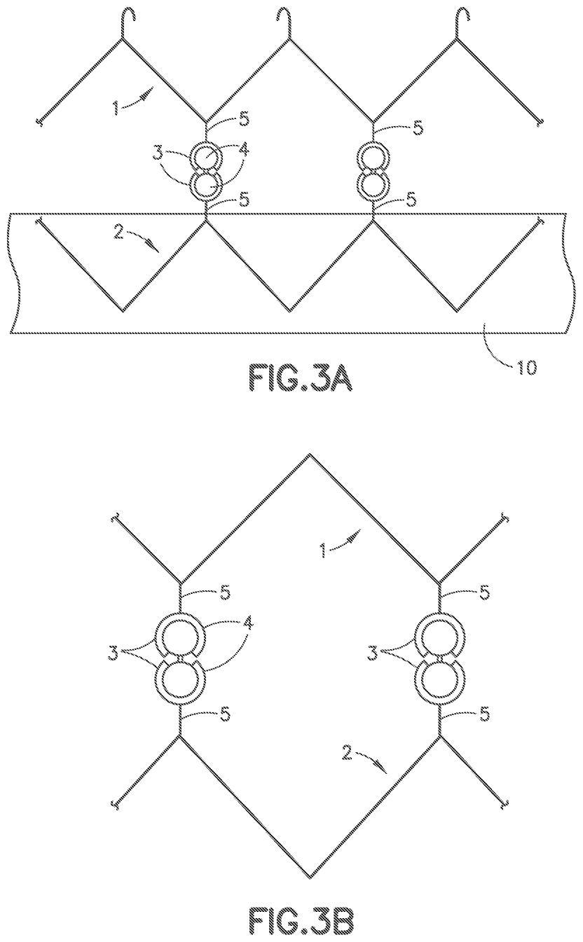

FIGS. 3A and 3B illustrates an alternative aspect having a single or double ball 4 and a socket 3 to form connectors optionally with a bar 5 on each side.

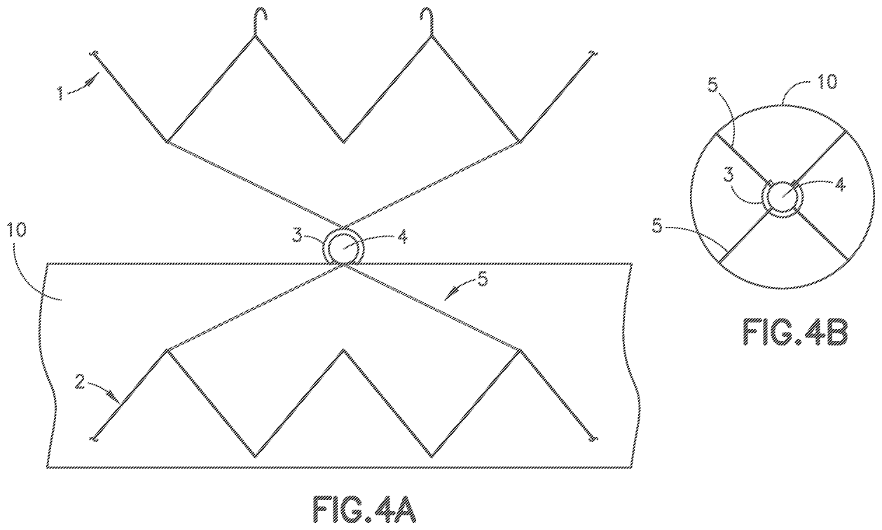

FIGS. 4A and 4B illustrate an alternative aspect with a single ball 4 disposed in the center of the lumen between the stent 1 and the connecting ring 2 with the ball 4 connected to the stent 1 and the connecting ring 2 by bars 5 and socket joint in the center of lumen, as shown most clearly in FIG. 4B.

FIG. 5 shows a top view of an exemplary alternative where a ball 4 is disposed on an apex of connecting ring 2 and is adapted to fit into a socket 3. The ball 4 is retained in socket 3, but is only free to rotate in the socket 3 substantially in a single plane, as shown by the arrows. In one aspect, the single plane is from medial to lateral, lateral to medial, or lateral to lateral. In this aspect, ball 4 is not free to rotate or move in a dorsal to ventral or ventral to dorsal plane.

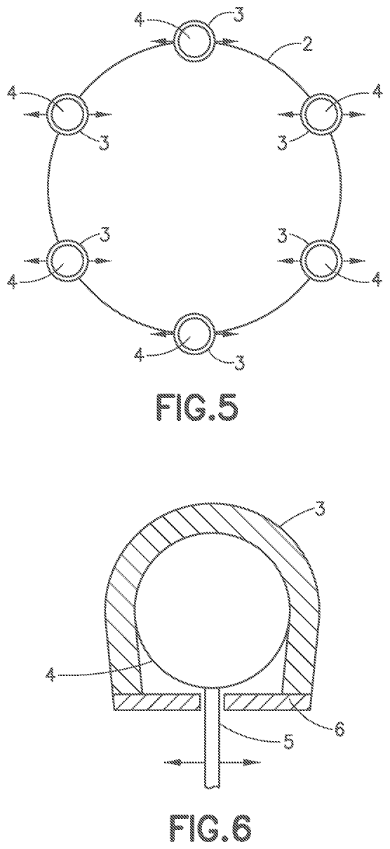

FIG. 6 illustrates a cross section of an exemplary ball 4 retained in a socket 3 with the ball 4 being connected to a bar 5. In this example, the bar 5 is limited to bending or moving in a single plane and is constrained by a channel 6. The channel 6 prevents the bar 5 and the ball 4 from substantially bending or moving outside the plane defined by channel 6.

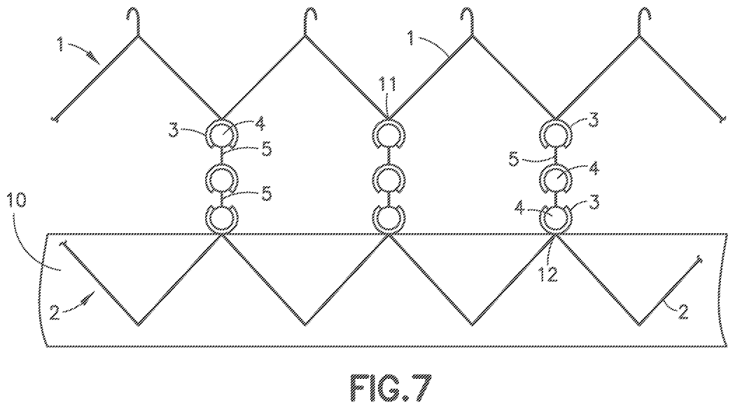

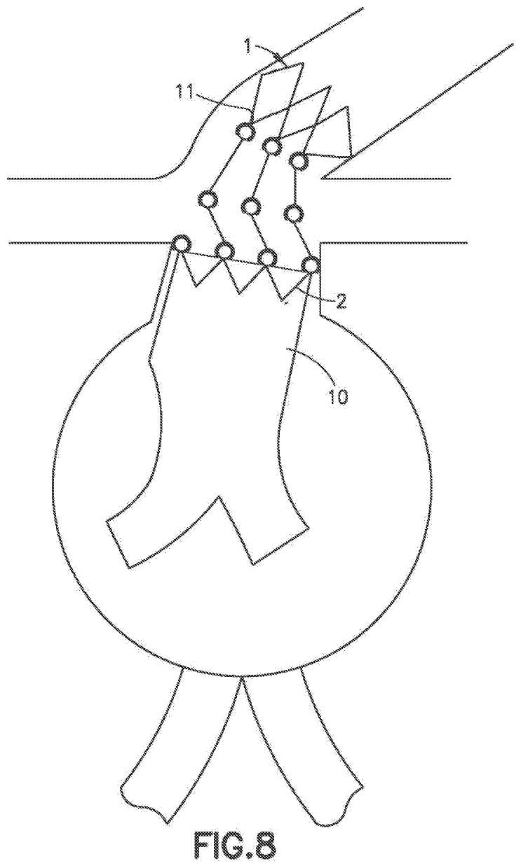

FIG. 7 schematically illustrates a cross section of a stent-graft device where apices 11 of a stent 1 are joined to apices 12 of a connecting ring 2 by plural struts or bars 5 and plural sockets 3 and balls 4. More particularly, an apex 11 of the stent 1 is opposed to an apex 12 of the connecting ring 2. Sockets 3 are connected respectively to the apices 11, 12. Two bars 5 are disposed between the aligned apices 11, 12. One of the bars 5 has a socket 3 and a ball 4 at the opposite ends, and the other of the bars 5 has balls 4 at both opposite ends. The stent-graft device of FIG. 7 thus provides three ball and socket joints between each of the interconnected apices 11, 12 of the stent 1 and the connecting ring 2. As shown in FIG. 8, the arrangement of three or more interconnected ball and socket joints allows the interconnection between the stent 1 and the connecting ring 2 to shorten on the inner side of a curve, thereby enabling the stent-graft device to conform more closely to the shape of the blood vessel.

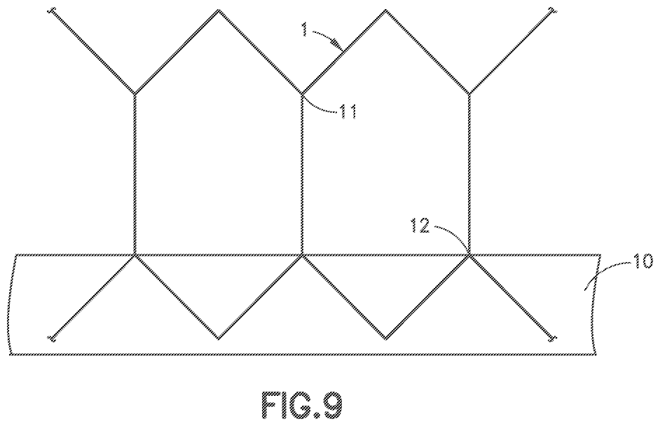

FIG. 9 illustrates an alternate embodiment of a stent-graft device with a stent 1 having apices 11 and a connecting ring 2 having apices 12. Unlike the previous embodiments, FIG. 9 does not have ball and socket connections. Rather, the stent-graft device of FIG. 9 has struts or bars 5 extending between and connecting the substantially aligned and/or opposed apices 11, 12. The struts or bars 5 are formed from a material that will exhibit a selected radially outward force to the graft 10 toward the blood vessel, thereby providing enhanced prevention of endoleaks.

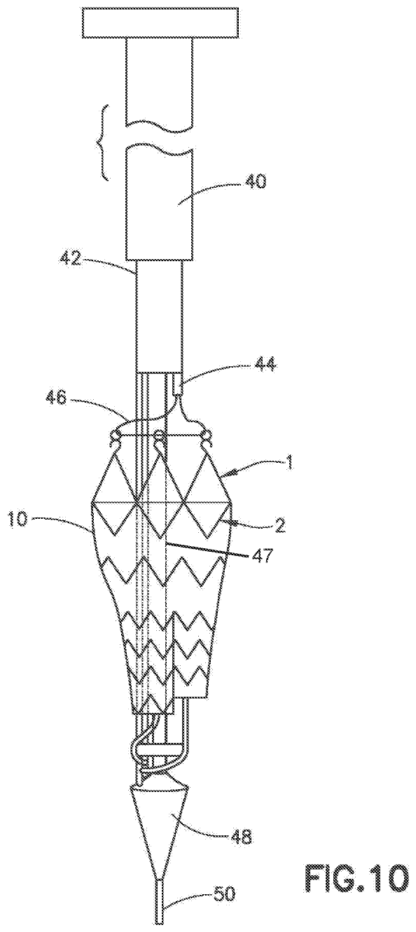

The stent-graft device described above and illustrated in the accompanying figures can be introduced into the targeted blood vessel in a conventional manner using an outer sheath 40 and a pusher catheter 42 illustrated schematically in FIG. 10. The pusher catheter 42 is dimensioned to be advanced axially within the outer sheath 40 and includes or accommodates an inner member 47 that may have internal or external channels for accommodating the various devices for positioning and deploying the stent-graft device. In this regard, a snare tube 44 may accommodate a snare loop 46 that passes through eyelets connected to the stent 1. A centering device 48 may be connected to an inner member that passes through the pusher catheter 42 and functions to center the stent-graft device within the blood vessel. A guide wire 50 may project axially from the centering device 48. An unillustrated side port may extend transversely from the illustrated outer sheath 40 to accommodate and permit further connections. Such further connections, for example, enable delivery of a saline solution and a radiographic contrast (dye). The radiographic contrast (dye) is used to perform angiograms of the aorta before deploying the device and an endograft after deployment. The side port also enables an angiogram to be taken partway through the procedure to see if everything intended by the procedure is being carried out as intended. The side port may have a three-way Luer Lok with locking lugs to permit the external connections. Such structures are known in the art. The transverse flange at the upper end of FIG. 10 defines a hub and has an aperture extending vertically through the outer sheath 40. The aperture may be closed by a self-sealing diaphragm or by an open/closed valving mechanism to permit the pusher catheter 42, wires and endograft components in while providing a liquid-tight seal to prevent bleeding. Outer sheaths and pusher catheters, such as the schematically illustrated outer sheath 40 and the schematically illustrated pusher catheter 42, are well known in the art, and the pusher catheter 42 can take any known forms for advancing the stent-graft device to an appropriate position in the targeted blood vessel by forces applied from positions external of the patient. The outer sheath 40 and the pusher catheter 42 can be referred to generally herein as delivery components.

It is understood that a ball, socket, and bar configuration can be of any suitable shape (e.g., rectangle, square, rhombus, cylinder, triangle, symmetrical, and non-symmetrical) and is not limited to spherical or rectangular shapes.

While the aspects described herein have been disclosed with reference to certain embodiments, numerous modifications, alterations, and changes to the described aspects are possible without departing from the sphere and scope of the present disclosure, as defined in the appended claims. Accordingly, it is intended that the present disclosure not be limited to the described aspects, but that it has the full scope defined by the language of the following claims, and equivalents thereof.

1-23. (canceled)

24. A stent-graft device having a flexible connection between a stent and a graft, the stent-graft device comprising:
a stent having stent apices; and
a connecting ring associated with a graft, the connecting ring flexibly coupled to the stent at the stent apices,
wherein the connecting ring is adapted to apply radial force to a blood vessel when the stent-graft device is deployed in the blood vessel.

25. The stent-graft device of claim 24, further comprising one or more tethers disposed between the stent and the graft for attaching the stent to the graft.

26. The stent-graft device of claim 25, wherein the one or more tethers comprise a suture, wherein the connecting ring is flexibly coupled to the stent by the suture.

27. The stent-graft device of claim 25, further comprising graft fixation tethers, wherein a first end of each of the graft fixation tethers is fixed to one or more locations on the connecting ring, and a second end of each of the graft fixation tethers is fixed to the graft.

28. The stent-graft device of claim 24, further comprising at least one delivery component for delivering the stent, the connecting ring and the graft through a single arterial puncture or incision in a blood vessel to a specified position in the blood vessel, the at least one delivery component having a first end that remains external of the blood vessel and a second end that is removably positioned in the blood vessel, the at least one delivery component being oriented relative to the stent, the connecting ring and the graft so that the stent apices are inserted through the single arterial puncture or incision in the blood vessel before insertion of an end of the stent opposite the stent apices.

29. The stent-graft device of claim 24, wherein the flexible connection between the stent and the connecting ring is adapted to bend substantially in one plane along a longitudinal axis.

30. The stent-graft device of claim 29, wherein the one plane contains the longitudinal axis of the stent and/or the graft.

31. The stent-graft device of claim 29, wherein the one plane extends from medial to lateral, lateral to medial, or lateral to lateral with respect to the stent and the graft.

32. The stent-graft device of claim 24, further comprising one or more bars disposed between the stent and the connecting ring for attaching the stent to the connecting ring.

33. The stent-graft device of claim 32, wherein one or more of the bars is capable of transferring radial force from the stent to the connecting ring when the stent-graft device is deployed in a blood vessel.

34. The stent-graft device of claim 33, wherein each of the bars has at least two pivoting joints between the corresponding stent apex and the connecting ring.

35. The stent-graft device of claim 32, further comprising at least one delivery component for delivering the stent, the connecting ring and the graft through a single arterial puncture or incision in a blood vessel to a specified position in the blood vessel, the at least one delivery component having a first end that remains external of the blood vessel and a second end that is removably positioned in the blood vessel, the at least one delivery component being oriented relative to the stent, the connecting ring and the graft so that the stent apices are inserted through the single arterial puncture or incision in the blood vessel before insertion of an end of the stent opposite the stent apices.

36. The stent-graft device of claim 32, wherein the bars are constrained such that the bars are capable of moving substantially in one plane with respect to the stent and the graft.

37. The stent-graft device of claim 36, wherein the one plane is from medial to lateral, lateral to medial, or lateral to lateral with respect to the stent and the graft.

38. The stent-graft device of claim 32, wherein the bars are constrained by channels disposed between the stent and the connecting ring.

39. The stent-graft device of claim 32, wherein one or more of the bars is capable of transferring radial force from the stent to the connecting ring when the stent-graft device is deployed in a blood vessel.

40. The stent-graft device of claim 24, wherein the stent and the connecting ring are coupled in an end-to-end and non-overlapping configuration.

41. A method of repairing an abdominal aortic aneurysm in a patient, comprising:
puncturing a blood vessel at an insertion site located above a diaphragm of the patient;
inserting a stent-graft system in the blood vessel at the insertion site, the stent-graft system comprising a graft, a connecting ring associated with the graft and adapted to apply radial force to a blood vessel when the stent-graft system is deployed in the blood vessel, and a stent having stent apices, the stent apices being connected pivotally or flexibly to the connecting ring; and
positioning the stent-graft system in the blood vessel of the patient while pivotally or flexibly moving the connecting ring relative to the stent apices to achieve an alignment of the connecting ring and the stent substantially conforming to an alignment of the blood vessel in proximity to the abdominal aortic aneurysm.

42. The method of claim 41, wherein the connecting ring is adapted to apply radial force to the blood vessel when the stent-graft system is deployed, securing the connecting ring to the blood vessel.
